(19) United States
(12) Patent Application Publication
(10) Pub. No.: US 2005/0164336 A1
(43) Pub. Date: Jul. 28, 2005

(54) Method for protein expression analysis

(76) Inventor: Peter James, Lund (SE)

Correspondence Address: AMERSHAM BIOSCIENCES;PATENT DEPARTMENT, 800 CENTENNIAL AVENUE, PISCATAWAY, NJ, 08855, US

(21) Appl. No.: 10/499,885

(22) PCT Filed: Dec. 16, 2002
(86) PCT No.: PCT/EP02/14328

(30) Foreign Application Priority Data
Dec. 22, 2001 (GB) 0130845.1

(51) Int. Cl. C12P 21/06; G01N 33/00

(52) U.S. Cl. 435/68.1; 436/86; 530/409

(57) ABSTRACT

The present invention is a method for analysis of at least peptides comprising extracting proteins from at least one set of cells; digesting the extracted proteins; derivatising the protein fragment mixture with an isotopically labelled reagent molecule; separating the protein fragment mixture by multi-dimensional chromatography; and analysing the protein fragment mixture by mass spectroscopy (MS) in parent ion or neutral loss scanning mode, thereby detecting or measuring the amounts of the labelled protein fragments. In a preferred embodiment, two sets of cells are combined in the method in order to compare the expression levels of two different states. Moreover, the invention relates to a kit for use in the present labelling method.

### TECHNICAL FIELD

The present invention relates to methods for the development of a fully automatable system for protein expression analysis using isotopic labelling of whole cell digests and their subsequent analysis by multi-dimensional chromatography and/or electrophoresis coupled to mass spectrometry and optionally database searching.

### BACKGROUND TO THE INVENTION

It was announced in early 2001 that the human genome had been sequenced. This marked the beginning of a new era for biological research. Essentially what was done was to determine the order of the four building blocks (nucleotides) that are joined together to form the pairs of DNA chains called the chromosomes. Humans have 46 of these, half of which we receive from our mothers, the other half from the father. The determination of the sequence of the human genome was ‘simple’ since there are only 46 molecules (albeit huge ones) made up of 4 building blocks or letters. Proteins have 20 building blocks, each of which can be modified or decorated after the protein is built. Hence, the study of the protein version of the genome, ‘proteomics’, must deal with 40,000 or more genes which can be arranged to give some 800,000 proteins (corresponding to some 10⁷ tryptic peptides), which in turn can be modified with over 300 different chemicals. Not only that, proteomics must also define which proteins are being produced in a certain type of cell at a specific time, how they are modified, where they are in the cell and with whom they are in contact and finally and most difficult, what is the function of the protein.

Today, some methods are available for such analyses and determinations. For instance, WO00/11208 discloses a method in which a protein is derivatised with an isotopically labelled molecule. The labelled protein is captured, digested, released and analysed by mass spectrometry.

WO01/74842 (Proteome Systems) teaches a method in which the desired protein sample is subjected to 2D-electrophoresis separation (2D-SDS), specific residues protected before digestion, and derivatisation with a labelled reagent and analysed by mass spectrometry. However, these methods have shown to be limited due to the 2D-electrophoresis, which does not allow a separation, which leads to a visualisation of all proteins. Many proteins are incompatible with this method, either being too small or too large, too acidic or alkaline, or just too insoluble. Membrane proteins, which is one of the most important groups of proteins, both physiologically and pharmaceutically, are completely underrepresented due to their tendency to aggregate and precipitate during various of the steps in 2D electrophoresis. Therefore, these proteins tend to be excluded from labelling and thus also from the analysis. In addition, the method disclosed in WO 01/74842 cannot be used in MS in parent ion-scanning mode, since the reagent described therein is not capable of generating any signature ions.

Further, WO 01/86306 (Purdue Research) relates to a method for protein identification in complex mixtures that utilises affinity selection of constituent proteolytic peptide fragments unique to a protein analyte. These “signature peptides”, which are low abundance amino acids such as Cys or Met, act as analytical surrogates for chemical capture of reagents. Mass spectrometric analysis of the proteolysed mixture permits identification of a protein in a complex sample without purifying the protein or obtaining its composite signature, since the use of “signature peptides” will reduce the complexity of the analysis. However, such “signature peptides” should not be confused with the signature ions required in MS in parent ion mode, which is not possible with the method disclosed in WO 01/86306.

Aebersold et al (American Genomic/Proteomic Technology (Aug. 2001), Vol. 1(1), p. 22-27) discloses isotope-coded affinity tag reagents for quantitative proteomics. However, this method requires the reduction in peptide complexity to be achieved by affinity purification and not by MS in parent ion-scanning mode. Likewise, Goodlet et al (Rapid Communications in MS, 2001, 15, 1214-1221) discloses a chemical tagging of proteins specific to Asp and Glu. The reagents are MS/MS stable, and cannot generate the specific fragment signature ions required in MS in parent ion-scanning mode. Another method which for the same reasons is not useful in parent ion-scanning mode either has been disclosed by Wang et al (Journal of Chromatography A, 924 (2001) 345-357), wherein chemical affinity chromatography is used to reduce sample complexity.

WO 02/48717 relates to an acid-labile isotope-coded extractant and its use in quantitative mass spectrometric analysis of protein mixtures. The reagents used in such method must be thiol specific and MS/MS stable. Thus, this method can not generate any signature fragment ions, and is consequently not useful in MS in parent ion-scanning mode.

Finally, Carr et al have described methods for following phosphate loss from phosphopeptides (Selective detection and sequencing of phosphopeptides at the femtomole level by mass spectrometry, Anal. Biochem. 239(2): 180-92, 1996). The method relies on the generation of a natural signature ion—79 m/z that is due to the loss of phosphate. The occurrence of phosphate can also be followed by the loss of phosphate as a neutral molecule using the neutral loss-scanning mode.

However, often it is desired to compare a cell in two different states, in order to determine the differences on the protein level. In these cases, a problem is to reduce the amount of data obtained by any one of the methods used today, in order to be able to focus on the relevant proteins. Thus, it would be advantageous to provide a method wherein the relevant proteins from different cell states are studied and compared in a better way.

Accordingly, an object of the invention is to provide a method solving the posed problems.

### SUMMARY OF THE INVENTION

The inventors have now developed a method, which meets the demands of the proteomics research society. Accordingly, in a first aspect the invention relates to a method as in claim 1 for labelling a protein or polypeptide mixture, which has been extracted from a set of cells, with an isotopically labelled reagent molecule, and analysing it with MS parent ion-scanning. In another preferred embodiment two different sets of cells, representing two different states, are analysed by the method, whereby each set of cells is labelled with different reagent molecules, allowing for a subtractive parent ion or neutral loss scanning. In another aspect the invention relates to the labelled reagent molecule, and in still another aspect the invention relates to a kit for use in the method of the invention, comprising the labelled reagent molecules.

Thus, this application describes the development of a non two-dimensional electrophoresis gel-based proteome analysis method. Essentially an isotopic labelling method is used to specifically label the N-terminal of all the peptides obtained from the digestion of a whole cell extract. In one embodiment, a first cell sample is labelled with the reagent and the second cell sample with the deuterated variant. The very complex peptide mixture (10⁷ peptides) is partially separated by two-dimensional chromatography/capillary zone electrophoresis and then analysed directly on-line by nano-electrospray mass spectrometry. The mixture may alternatively be collected in such a manner as to allow a subsequent off-line analysis such as by MALDI mass spectrometry. Therefore, the inventors have synthesised the reagent with a thioether bridge connecting to an isotopically labelled amine moiety. The thioether bridge is chemically very stable, however, in the gas phase it fragments easily to give a daughter ion at a unique mass. By parent ion-scanning, the inventors can detect only those peptides that contain the unique mass label. Since there are two masses, light and heavy from the deuterated and non-deuterated reagents, we can set the mass spectrometer to sequence only those peptides whose expression level is changing by a set factor. The method can be tuned for detection of peptides by neutral loss scanning by reducing the basic nature of the leaving group.

Thus the number of peptides to be analysed drops from 10⁷ to around 10³ depending on the system. In contrast to 2D PAGE technology, all proteins are represented, including membrane, large and small and extreme pI proteins. The method can easily be automated and be a valuable alternative to the slower and limited methods in current use.

In one embodiment, the present invention provides a measure of the expression level of the protein or polypeptide labelled. To this end the present invention is especially advantageous, since for the first time it enables to filter thousands of proteins that are not changing their expression levels.

Thus, in this application the basis for a novel method for analysing all the proteins in a cell and their modifications is described. Unlike current methods which firstly separate proteins according to their charge and then by size, this method chops all the proteins in a cell down into small pieces (peptides) before separating them according to their charge and fat solubility. It will allow the analysis of all those proteins which cannot easily be found using conventional techniques such as membrane, very large or small or highly charged proteins. Eventually one may be able to replace the cumbersome methods in use today with a simple, easily automated computerised method and give many more scientists, and more importantly clinicians access to a very powerful research tool.

Other objects and advantages of the present invention will appear from the detailed description that follows.

### SHORT DESCRIPTION OF THE DRAWINGS

FIG. 1 shows the thioether-bridged isotopic labels H4S and D4S.

FIG. 2 shows a MS/MS spectrum of a peptide with H4S covalently linked to the N-terminal.

FIG. 3 shows the construction of a parent ion-scanning mass spectrometer.

FIG. 4 shows the principle behind protein expression analysis by subtractive parent-ion scanning.

### DEFINITIONS

By “a biomolecule” is meant any of several occurring biomolecules, such as proteins, polypeptides, peptides, nucleic acids, fatty acids, carbohydrates, in an organism, such as a human.

By “a set of cells” is meant a number of cells, for example only one cell, or a large number of cells, which have been isolated from a relevant organism, such as a human, in a specific state.

By “a reagent molecule” is meant a molecule having the ability to covalently bind to a specific site in a protein, thereby, if labelled, being used to detect the bound protein in an analysis.

By “a binder part” is meant a part of the reagent molecule having the ability to bind to a specific site on a desired protein.

By “a labelled part” is meant a part of the reagent molecule comprising a label, which is possible to detect, by some subsequent analysis, such as mass spectroscopy.

By “a bridge part” is meant a part of the reagent molecule linking the label and the binder part, thereby, after cleavage of the bridge part, allowing detection of a unique labelled mass marker in a subsequent analysis, such as mass spectroscopy.

By “derivatising” a mixture of protein fragments with a reagent molecule is meant to allow the reagent to covalently bind to specific sites in the protein fragments.

### DETAILED DESCRIPTION OF THE INVENTION

Accordingly, a first aspect the invention is a method for protein analysis comprising the steps of:
(a) extracting at least one protein or a polypeptide from at least one set of cells;
(b) digesting the extracted protein/polypeptide, thereby obtaining a mixture of peptides or protein-fragments;
(c) derivatising the peptide mixture with an isotopically labelled reagent molecule, whereby the reagent binds to specific sites of the protein fragments;
(d) separating the peptides of the mixture by multi-dimensional chromatography;
(e) analysing the peptide mixture by mass spectroscopy (MS), wherein a signature ion specific to each peptide is generated and the amounts of labelled peptides are detected in parent ion-scanning or neutral loss scanning mode. For neutral loss scanning, a less basic leaving group is chosen than in parent ion-scanning.

Even though the present method relates to protein analysis, the skilled in this field could easily adapt the method to the analysis of any other biomolecule, the nature of which renders it suitable for the procedure outlined herein. Accordingly, the present invention also embraces the method above for labelling at least one biomolecule and determining the amount thereof. As the skilled person in this field will realise, in order to be useful in MS in parent ion or neutral loss scanning mode, the present labelling reagent is MS/MS fragile. In the best embodiment at present, the labelling reagent comprises a binder part, a bridge part and a label part, wherein the bridge part is a thioether bridge.

Thus, the method according to the present invention does not require any prepurification using affinity steps or chemical capture, such as in the prior art methods discussed above. Further, since the present method utilises MS in parent ion or neutral loss scanning mode, the mass spectrometer can be setup so that only those peptide ions coming from proteins changing their expression levels can be detected. Since each protein is represented by multiple peptides, the danger of missing a protein or a post-translational modification is greatly reduced. In the prior art methods relying on a unique peptide to represent a protein, as is the case for the affinity purification or labelling using labelling of rare amino acids, the chances of missing that peptide due to coelution with multiple other peptides is great.

In yet another preferred embodiment of the invention proteins or peptides are extracted from two sets of cells, whereby each set of cells are labelled with a different reagent. Hereby, two different states are compared and/or their expression levels determined, as the two samples are mixed and subjected to subtractive parent ion-scanning. This is a preferred embodiment, which allows the comparison of two different states.

In a specific embodiment, the labelled peptide mixtures are combined prior to step (d).

In one embodiment, the sample provided in step (a) has been obtained by mechanical or chemical cell disintegration and centrifugation. In another embodiment, the sample provided in step (a) comprises membrane or membrane-associated protein(s). This embodiment is especially advantageous, since such proteins have shown to be quite problematic to label in the prior art. As mentioned above, the dual function of both hydrophilicity and hydrophobicity of such proteins often results in self-aggregation thereof, which in turn makes them inaccessible for any further analysis. In a specific embodiment of the present invention, the first digest of choice is carried out in formic acid, which dissolves virtually all proteins. After the digest, the acid is removed and the smaller peptides are all soluble in chaotrope solutions like urea where they can easily and efficiently be digested with enzymes into small peptides, most of which do not show the tendency of the intact protein to aggregate. Thus, according to the present invention, even though a few peptides from a protein may indeed aggregate, there will still be at least 50% minimum who do not. Accordingly, the present method has shown to be more advantageous than the prior art methods in the context of membrane and/or membrane associated proteins.

The labelling reagent used in step (c) above will for example label the N-terminal amino acids by virtue of its reaction with free amino groups. Accordingly, it is necessary to pre-treat the proteins to block binding of the labelling reagent to amino groups present on internal amino acid residues, especially lysine. If the epsilon groups on lysine were not blocked, then the labelling reagent would also bind to all free amino groups on the lysines making it difficult to interpret the amino acid sequence of the labelled peptide. Thus, in one embodiment, a succinylation of protein(s) is performed before step (b). In a specific embodiment, the protecting agent used in step (b) is succinic anhydride, but the protecting agent could be any suitable protecting agent that fulfils the above-described function. For example, N-hydroxysuccinimide can be added, e.g. at a pH of about 8. However, this protecting agent adds not only to lysine residues but also to tyrosine and serine/threonine. Thus, for such agents, a further step will be required, wherein these side-reactions are removed. This further step should be accomplished after the derivatisation of step (c), but before the peptide separation of step (d), and can for example be an addition of hydroxylamine (0.2 M), pH 8, for about 30 minutes. The skilled in this field is familiar with the art of protection and deprotection of amino acids and will be capable of selecting the appropriate conditions for each situation.

The cleaving in step (b) can be an enzymatic digestion, such as with an enzyme, such as a protease (e.g. trypsin, V8 protease, such as Staphylococcus aureus V8 protease, LysC, AspN etc) or a glycosidase, or a chemical digestion, such as with cyanogen bromide. However, as regards membrane and/or membrane associated proteins, due to their compact structure and tendency to aggregate when denatured, enzyme digestions can be found to be inefficient. In one embodiment which is especially advantageous for membrane and/or membrane proteins, the cleaving in step (b) is an enzymatic digestion preceded by addition of a digestive chemical, such as cyanogen bromide. More specifically, the present inventors have used a scheme wherein the proteins are first digested with cyanogen bromide in a powerful solvent, such as 70% formic or trifluoroacetic acid, with or without hexafluoropropanol. This generates medium sized fragments which can be readily solubilised by a conventional method, e.g. in 1% SDS, before dilution to about 0.01% and digestion with LysC protease. In an alternative embodiment acid-based cleavages are used, as reported by the group of Tsugita (Kamo et al. 1998 and Kawakami et al. 1997). Thus, in one embodiment, the cleaving in step (b) is a serine/threonine cleavage with a fluorinated acid. In a detailed embodiment, site specific cleavage at serine and threonine is carried out in peptides and proteins with S-ethyltrifluorothioacetate vapour as well as at aspartic acid residues by exposure to 0.2% heptafluorobutyric acid vapour at 90° C. Such a serine/threonine cleavage method is advantageous, since Ser and especially Thr are found often in transmembrane segments. In summary, the skilled in this field can select the most appropriate method to cleave the proteins in the sample depending on factors such as the source of the sample, the purpose of the labelling etc. The digested proteins obtained according to the present invention are much easier to handle since physicochemically they are much simpler. Thus, an essential advantage with the present invention is that the separation of peptides obtained according to the invention can be selected to pick out virtually any one or ones of those present in the original sample as proteins, since the present digestion will be essentially total. Accordingly, in the step of separation and the subsequent labelling, any one of all possible peptides (fragments of proteins) can be treated, even cysteine-containing peptides, as will be discussed in more detail below. This should be compared to the prior art methods, wherein proteins can be hidden or concealed due e.g. to self-aggregation. Prior methods required the separation of intact proteins and could not deal with peptide digests without losing the quantitation aspect. The present method of cleavage provides homogenous peptides, which can be separated without the problems associated with proteins have multiple domains (hydrophobic and hydrophilic) which cause them to run at multiple positions. The present digestion method also allows the analysis of proteins that are otherwise completely insoluble or are parts of large complexes, which can not be easily separated, especially cytoskeletal aggregates or proteoglycans.

According to one embodiment the reagent molecule of the invention is an amine derivative. In another embodiment the reagent molecule of the invention is constituted in order to have the ability to covalently modify the N-termini of peptides having a basic moiety. Thus, the binder part of the labelling reagent can in a preferred embodiment be any moiety, which reacts with an N-terminal amino group. Further, in a preferred embodiment, the labelling reagent comprises a thioether bridge, being a very stable chemical group, however having the ability to easily break in the gas phase (as in the MS).

Advantageously, the present invention utilises labels that can be produced in two or more forms which confer the ability to distinguish the different forms of labelled reagents and the peptides to which they are linked by mass, but which importantly do not affect the ionisation efficiency of the peptides to which they are linked when subject to mass spectrometry. In one embodiment of the present method, step (c) is treating with a reagent available in different forms that can be distinguished on the basis of mass. Thus, the reagent is selected from the group that consists of C12/C14; H/D; C135/37; positively charged aromatic amines; positively charged tertiary quaternary amines; and phosphorous-based compounds.

In an illustrative embodiment, which is preferred, two samples are provided in step (a), one of which is treated with H4S, and the other one with D4S.

For the preparation of precursor for H4S and D4S, and for the preparation of the H4S/D4S-reagent of the invention, see the example section of this application.

Hereby, a unique mass marker is provided, since H4S gives rise to a peak in the MS spectrum corresponding to 106 m/z and D4S gives rise to a peak corresponding to 110 m/z. However, as the skilled in this field easily realises, virtually any other pair of heavy/light isotopes can be used to this end, as long as unique mass markers are provided, which allows the use of a subtractive parent ion-scanning.

In one embodiment of the present method, the separation according to step (d) is by multi-dimensional chromatography. In another embodiment of the present method, standard reverse phase HPLC is used to separate the majority of the peptides. In a specific embodiment which is efficient if it is desired to get the most hydrophobic peptides, a hydrophilic interaction chromatography (HILIC) approach is used. Alternatively, a first dimension separation can be carried out by ion exchange in the presence of a detergent such as octylglucoside as demonstrated previously (James P, Inui M, Tada M, Chiesi M, Carafoli E. The nature and site of phospholamban regulation of the Ca2+ pump of sarcoplasmic reticulum. Nature. 1989 Nov 2;342 (6245):90-2). The detergent is then easily removed prior to RP-HPLC-MS analysis by using a normal phase precolumn. Alternative combinations could also include the various forms of capillary zone electrophoresis, size exclusion chromatography or a specific affinity purification step.

The total amount of protein needed to observe all peptides in a cell and the degree of separation needed are important parameters to find. Accordingly, if one to start with assumes that the maximum sensitivity level for peptide detection and MS/MS is 1 fmol. There are thus 6×10⁻²³ moles of this protein per cell, therefore 1.6×10⁷ cells are needed assuming a number which is equivalent to 0.25 mg of protein. Thus, the first dimension separation will have to be carried out on a 1 mm column at the analytical level. The second dimension chromatography can then be done with a 150 μm column. Given the human genome is assumed to have 30,000 genes, of which 10% are expressed in any one cell line at a given time, and assuming there are on average 20 variants of each protein due to alternative splicing, post-translational modification etc., there will be approximately 200,000 tryptic peptides per cell given an average protein molecular weight of 50 kDa. In order to avoid too much signal suppression, one should aim to have a separation method that produces individual spectra containing 10 peptides or less. Given 10 fractions from the first dimension and a second dimension flow rate of 200 nl/min, the peak width will be about 5 sec. Thus a single gradient will have to be around 2.7 hours if a maximum of 10 peptides are to be observed per scan on average, giving a total analysis time of 27 hours.

The inventors have built a two-dimensional HPLC system based on that described by the group of Stahl et al. 1999 (Anal Chem 1995 Dec 15;67(24):4549-56. A microscale electrospray interface for on-line, capillary liquid chromatography/tandem mass spectrometry of complex peptide mixtures. Davis M T, Stahl D C, Hefta S A, Lee T D.). Since there are no commercially viable instruments capable of operating in the low nanolitre per minute range without flow splitting, the inventors constructed a nanoflowed HPLC based on the design that was built in Zurich. The first dimension is carried out using a commercial device at moderately high flow rates (50 μl/min) and the second dimension is carried out using the nanoflow design of the inventors run at 1-200 nl/min in a dynamic fashion according the number of peptides eluting. The first dimension can use strong anion exchange chromatography at pH 3 to generate 10-20 fractions that then are collected in an autosampler and then separated by reverse phase C-18 based chromatography coupled to the mass spectrometer.

According to the invention, the detection or measuring is by mass spectroscopy (MS). More specifically, parent ion-scanning (Anal Chem 1996 Feb 1;68(3):527-33. Parent ion scans of unseparated peptide mixtures. Wilm M, Neubauer G, Mann M. and Carr et al. 1993, Anal Chem 1993 Apr 1;65(7):877-84 Collisional fragmentation of glycopeptides by electrospray ionisation LC/MS and LC/MS/MS: methods for selective detection of glycopeptides in protein digests. Huddleston M J, Bean M F, Carr S A.) is used to detect the unique mass marker labels of the invention.

In another embodiment of the present method, the detected label is present on a cysteine-containing peptide. Accordingly, contrary to the prior art, such as Gygi S P, Rist B, Gerber S A, Turecek F, Gelb M H, Aebersold R. Related Quantitative analysis of complex protein mixtures using isotope-coded affinity tags. Nat Biotechnol. 1999 Oct; 17(10):994-9, the present invention provides a method, which is useful on any peptide or protein, regardless of its cysteine content. Since about 20-30% of the proteins of the human genome contains the amino acid cysteine, this is an essential advantage of the invention, which broadens its applicability and makes it a more general method than the ones previously disclosed. Also, the number of labelled peptides obtained from a cysteine labelled protein is of the order of 1-2. If the mass spectrometer is analysing a coeluting peptide from another peptide during the time another is eluting, one protein will be excluded from the analysis. Since in the invention, a protein typically generates 10-200 peptides, there are numerous other possibilities to analyse a peptide arising from this protein, thus the chances that is lost is vanishingly small.

In yet another embodiment, the present method comprises the method described above, which further comprises the step of identifying the amino acid sequence of at least one of the labelled peptides.

In one embodiment, amino acid sequence identification step is by mass spectral analysis using an ion trap spectrometer or a quadrupole time of flight (TOF) instrument. However, as is realised by the skilled in this field, any MS instrument capable of carrying out and measuring peptide fragmentation spectra can be used to this end.

Moreover, the amino acid identification may be followed by a data base search, in order to find homologues, or other relevant information, to the identified sequence. This may be done in order to assign a probable function for the identified sequence.

There are two approaches to accumulating the data. Either a flow splitter can be installed before the MS so that half goes the MS and half to a fraction collector. In the first method, post-processing, the MS spectra are analysed after the run and the peptides changing their expression levels are retrieved from the appropriate fractions for MS/MS analysis. The second method, dynamic data-dependant analysis (Davis et al. 1995), evaluates the H4SD4S ratio on-the-fly, processing the spectrum immediately and then automatically carrying out MS/MS if the ratio shows an appropriate change. Initial experiments using a Finnigan triple quadrupole mass spectrometer and a self-programmed Instrument Control Language program showed that this is possible.

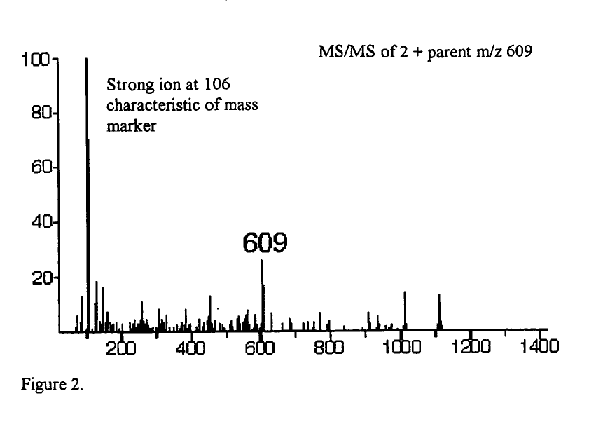

An alternative method developed to allow the quantitation of multiple proteins in a single spot from a two-dimensional was recently described (Münchbach M, Quadroni M, Miotto G. James P. Quantitation and facilitated de novo sequencing of proteins by isotopic N-terminal labelling of peptides with a fragmentation-directing moiety. Anal Chem. 2000 Sep 1;72(17):4047-57), which can be extended to direct labelling of peptides released from a digestion of a whole cell protein extract. The isotopic labelling allows a quantitative analysis of protein expression levels as well as facilitating the identification of the peptides generated (see FIG. 2).

In an advantageous embodiment, the first labelling reagent comprises a light isotopic label and the second labelling reagent comprises a heavy isotopic label, or vice versa. Labelling reagents can be selected from the group discussed above in relation to the first aspect of the invention. Thus, in a specific embodiment, said first and second labelling reagents are H4S and D4S.

Another aspect of the present invention is a reagent molecule for use in labelling a peptide or a protein for expression analysis comprising at least a binder part, a bridge part and a labelled part. In one embodiment the binder part has the ability to covalently modify the N-termini of a peptide having a basic moiety. In another embodiment the bridge part is a thioether bridge. In still another embodiment the labelled part comprises at least one hydrogen/deuterium atom. In yet another preferred embodiment the molecule is N-succinimidyl-2-(4-pyridylmethylthio)-acetate (referred to as H4S), and its deuterated variant is N-succinimidyl-2-[4-(2,3,5,6-tetradeuterio-pyridyl)]-methylthioacetate (referred to as D4S).

The reagent molecule may be any molecule, as long as it exhibits some necessary features. The thioether bridge, or any equivalent alternative, is important in order for the dissociation to occur in the gas phase. Further, the labelled part of the molecule is preferably positively charged, or at least electrophile, in order to make it possible to cleave the thioether bridge in the gas phase. Moreover, the labelled part of the molecule may comprise one or more metal atoms, such as Sn, in order to provide it with the desired chemical properties. Furthermore, it must allow the detection of at least one unique mass marker. Thus, it must comprise at least one atom, which is possible to substitute for an isotopic alternative, such as hydrogen/deuterium (H/D). Preferably, the labelled part comprises 2-6 isotopically substitutable atoms since large numbers of deuteriums can affect the chromatographic behaviour of the modified peptides causing them to elute at different times precluding an on-line dynamic analysis. Still further, a mix of reagent molecules having a varying amount of isotopically substitutable atoms, such as two H/D, three H/D, four H/D, five H/D and six H/D may be used, in order to improve the speed of analysis, since multiplexing can be carried out allowing 1, 2, 3 and 4 or more cells to be analysed in a single MS-chromatographic run.

Further, it must have the ability to bind to the desired biomolecule. For example, the reagent molecule may be designed to be able to bind to the N-terminal of peptides, as discussed above.

Thus, in a preferred embodiment, the reagent molecule of the invention is N-succinimidyl-2-(4-pyridylmethylthio)-acetate, which reagent hereafter is referred to as H4S. Its deuterated variant, N-succinimidyl-2-[4-(2,3,5,6-tetradeuterio-pyridyl)]-methylthioacetate, is referred to as D4S. As discussed above, modifications of this molecule especially in respect of the labelled part and the binder part (such as for different biomolecules) may be made, as long as it exhibits the necessary features. Furthermore, the different functional parts of the reagent molecule (binder part, labelled part, bridge part) must not necessarily be distinct from each other, as long as the molecule displays the desired properties.

Yet another aspect of the invention is a kit for use in labelling a mixture of protein fragments for parent ion-scanning, comprising, in separate compartments, H4S and D4S as defined above. The kit may further comprise other components necessary or favourable to use in combination with H4S and D4S. Such components may easily be read out from the description as outlined here.

Still another aspect of the invention is the use of at least one reagent molecule as described above for labelling a mixture of protein fragments for subsequent parent ion-scanning.

The present invention can be used in a wide variety of applications, such as for example to identify peptides presented by a major histocompatibility complex (MHC) molecule.

Another application where the present method is useful is for the analysis of peptides being carried around or in solution in body fluids such as cerebro-spinal and synovial fluids as well as in urine and blood serum. Accordingly, the method according to the present invention can be used e.g. in diagnosis of diseases.

As discussed above, the use of two or more labelling reagents with different labels allows a determination of relative amounts of proteins in two or more different samples. In particular, the labelling techniques of the present invention may be used to compare protein expression in two different cells. The two different cells may for example be cells of the same type but under different conditions (or states), or they may be cells of a different type (under the same or different conditions). Thus, by way of example, a first cell may be treated with an agonist and a second cell untreated, and the expression of one or more proteins in each cell compared.

The two conditions could also be cells resting versus cells induced or treated in some manner. Often, differential expression in cells under different conditions can provide useful information on the activity in the cells.

Thus, in an advantageous embodiment of the present invention, the protein-fragment mixture is analysed at a first frequency, thereby generating a first set of cells, and then at a second frequency, thereby generating a second set of cells, followed by an inversion of the intensity values of the second frequency and adding them to the first, whereby a difference spectrum is generated. The second frequency is usually higher than the first, and the analysis is a scanning, such as a parent ion-scanning or a neutral loss scanning. Accordingly, a specific embodiment is a method, wherein the protein-fragment mixture is analysed by (i) scanning at 106 m/z, thereby generating a spectrum of the first set of cells, (ii) scanning at 110 m/z, thereby generating a spectrum of the second set of cells, (iii) inverting the intensity values of the 110 m/z scan and adding them to the 106 m/z scan, thereby generating a difference spectrum. Another embodiment a method, wherein the protein-fragment mixture is analysed by (i) neutral loss scanning at 105 m/z, thereby generating a spectrum of the first set of cells, (ii) neutral loss scanning at 109 m/z, thereby generating a spectrum of the second set of cells, (iii) inverting the intensity values of the 109 m/z loss scan and adding them to the 105 m/z scan, thereby generating a difference spectrum.

In one specific embodiment, the digestion step of the present method is performed in a device for protein and/or peptide concentration in a sample, which device comprises electroconcentration means comprising a funnel shaped cavity with a wide end and a narrow end; at least two electrodes, one electrode being positioned near to said wide end and one electrode being positioned nearer to said narrow end; and one or more protein and/or peptide capture means; wherein said capture means is located between said narrow end and said one electrode positioned near said narrow end.

In the preferred embodiment, the present device is presented as an assembly held together by a seal. During use, the whole device is preferably held within a pressurised container at around 2-3 bar to prevent the formation of bubbles which otherwise might form during electrophoresis from blocking the passages and stopping the current flow. Accordingly, this device may be used in a method for concentrating a protein and/or a peptide in a sample, comprising the steps of providing a sample which comprises proteins and/or peptides and a digestive agent in an electrophoresis device, wherein the electroelution bath is present in an essentially funnel shaped cavity; applying a voltage between at least two electrodes located on each side of said electroelution bath to pass peptides towards a capture means located between the narrow end of said funnel shaped cavity and the electrode positioned nearer said narrow end; changing the direction of the voltage at least once to provide oscillations enabling both positively charged and negatively charged peptides to contact the capture means, and collecting concentrated peptides from the capture means. The device described above has been presented under the denotation DigTag™.

Hereby, the digestion according to step (b) of the invention may be performed in an alternative way.

The invention will now be described with reference to the following examples, which only are intended to exemplify the invention, and not to limit the scope of the invention as defined by the appended claims. All references given below and elsewhere in the present specification are hereby included herein by reference.

### EXAMPLES

### Example 1

### Preparation of Precursor (Pyridylethanthiol)

Nictonic acid (either D4 or H4) was converted to the acylchloride with thionylchloride. The extra thionyl chloride was removed by gentle heating and the solution used directly for an Arndt-Eistert reaction. An ice-cold solution of diazomethane in ether was added to the precooled acylchloride and slowly allowed to warm to room temperature. The solution was left overnight under nitrogen with vigorous shaking before adding silver benzoate. Distillation gave fairly pure (>90%) pyridylethanoic acid. This was subsequently reduced with lithium aluminium hydride to give pyridylethanol. This was treated with thionylbromide followed by sodium hydrosulphide to give pyridylethanthiol. This could be stored indefinitely and was the starting reagent for the synthesis of the protein modification reagent, which was carried out fresh each time.

### Example 2

### Preparation of H4S/D4S Reagent

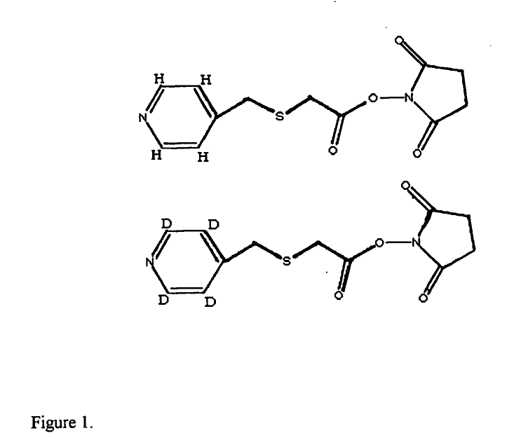

An appropriate amount of pyridylethanthiol of example 1 was added to an equimolar amount of iodoacetic acid. The resultant solution was mixed with one equivalent of dicyclohexylcarbodiimide for 6 hours at room temperature. One equivalent of N-hydroxyccinimide were added to the solution and stirred over night at room temperature. The precipitate was recovered by filtration and purified by recrystallisation from ethylacetate, to provide D4S or H4S (FIG. 1). The overall yield from nicotinic acid was low, ca. 10%.

### Example 3

### Isotopic Labelling and Detection by Parent Ion-Scanning

The basis of the isotopic labelling method is the use of isotopically labelled amine derivatives to covalently modify the N-termini of peptides with a basic moiety that allows one to distinguish between two sets of peptides. The inventors have described a preliminary set of reagents (Münchbach et al. 2000) that have been used to quantify and identify multiple proteins isolated by 1- and 2D gel electrophoresis. The inventors have recently developed a new set of reagents, the structures of which are shown in FIG. 1.

The basic feature of this reagent is that it can be specifically attached to the N-terminus of peptides generated from whole cell digests as the inventors have already shown for less complex mixtures. The proteins are extracted from the cell with 1% SDS and are succinylated. The proteins are then digested with cyanogen bromide and then Staphylococcus aureus V8 protease at pH 4. The peptide mixture is then derivatised with the reagent, either H4S or D4S and then the mixture is treated briefly with hydroxylamine to remove any side-reactions of succinylation or the isotopic reagent on Ser/Thr or Tyr. The D4S and H4S labelled samples from the two cell states are then mixed and the peptide mixture separated by 2D chromatography.

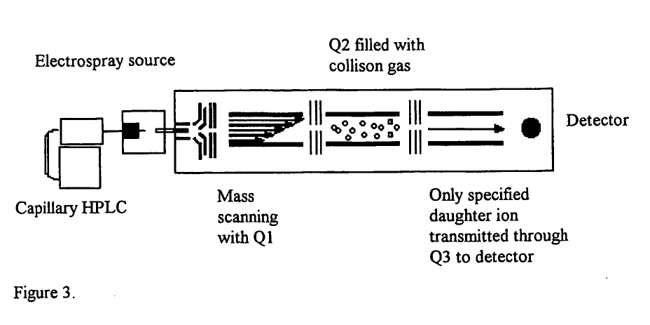

The N-terminally derivatised peptides are chemically very stable. However, in the gas phase the thioether bond of the derivative fragments easily generates a strong ion signal at 106 m/z as one can see in FIG. 2. This allows one to carry out parent ion-scanning by setting the MS to monitor the signal at 106 to detect the peptides giving rise to this signal. In this way one can selectively detect the H4S labelled peptides from cell state 1 (see FIG. 3). Parent ion-scanning has been previously used for the selective detection of N- and O-linked carbohydrates (ion at 204 m/z, Carr et al. 1993) and phosphorylated serine or threonine (ions at −80 and 98 m/z, Carr et al. 1996)

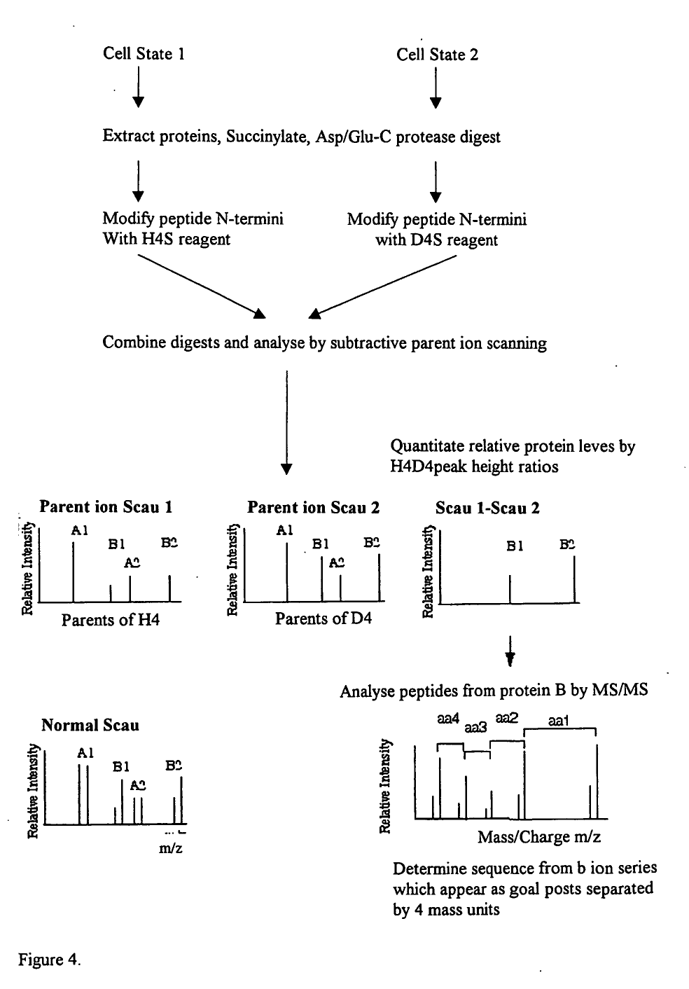

Thus by alternatively scanning for the parents of 106 (the H4S labelled peptides from state 1 and then for the parents of 110 (the D4S labelled peptides from state 2) one can visualise the relative expression levels of each peptide pair. This can be done dynamically by taking parents of H4S in scan 1, then parents of D4S in scan 2, inverting the intensity values of scan 2 and adding this to scan 1 to generate the difference spectrum as shown in FIG. 4. Only those peptides increasing or decreasing by a specified value will be observed in the difference spectrum, thus greatly simplifying the data. Instead of 200,000 peptides, only the 10,000 peptides (ca. 200 proteins) that are changing their expression levels will be observed and can be dynamically scheduled for MS/MS analysis on the fly.

1. A method for labelling at least one protein or a polypeptide and determining the amount of labelled protein/polypeptide comprising the steps of:
(a) extracting at least one protein or a polypeptide from at least one set of cells;
(b) digesting the extracted protein/polypeptide, thereby obtaining a mixture of peptides or protein-fragments;
(c) derivatising the peptide mixture obtained with an isotopically labelled MS/MS fragile reagent molecule, whereby the reagent binds to specific sites of the protein-fragments;
(d) separating the peptides of the mixture by multi-dimensional chromatography; and
(e) analysing the peptide mixture by mass spectroscopy (MS), wherein a signature ion specific to each peptide is generated and the amounts of labelled peptides are detected in parent ion or neutral loss scanning mode.

2. The method of claim 1, wherein the extraction is performed by using a buffer comprising sodium dodecyl sulphate, e.g. about 1% SDS.

3. The method of claim 1, wherein the extracted proteins/polypeptides are succinylated before digestion.

4. The method of claim 1, wherein the extracted proteins/polypeptides comprise membrane and/or membrane associated proteins.

5. The method of claim 1, wherein step (e) provides a measure of the expression level of the protein/polypeptide labelled.

6. The method of claim 1, wherein the digestion is performed by first using cyanogen bromide and then V8 protease at a pH of 4 to 5, or LysC protease at a pH of 7 to 9.

7. The method of claim 1, wherein the reagent molecule includes at least a binder part, a bridge part and a label part, wherein the bridge part is a thioether bridge.

8. The method of claim 7, wherein the binder part of the reagent molecule is an amine derivative.

9. The method of claim 8, wherein the amine derivative reagent has the ability to covalently modify the N-termini of peptides having a basic moiety.

10. The method of claim 7, wherein the label is distinguished on the basis of mass, and wherein the isotopic label includes at least one atom selected from the group consisting of C12/C14, H/D and C135/C137.

11. The method of claim 7, wherein the reagent is N-succinimidyl-2-(4-pyridylmethylthio)-acetate, and/or N-succinimidyl-2-[4-(2,3,5,6-tetradeuterio-pyridyl)]-methylthioacetate.

12. The method of claim 1, wherein the mixture after step (c) is treated with hydroxylamine.

13. The method of claim 1, wherein in the step (d) two-dimensional chromatography, wherein the first dimension uses anion exchange chromatography, and the second dimension uses reverse phase chromatography (RPC).

14. The method of claim 13, wherein the flow rate of the first dimension is in the interval from 1 to 100 μl/min and the flow rate of the second dimension is in the interval from 1 to 200 nl/min.

15. The method of claim 1, wherein the detected label is present on a cysteine containing peptide.

16. The method of claim 1, wherein the mass spectrometry analysis is performed at 106 and/or 110 m/z.

17. The method of claim 1, wherein the sample in step (e) is divided in two fractions, whereby one is directed to MS and one to a fraction collector.

18. The method of claim 1, wherein proteins/polypeptides are extracted from two sets of cells, and each set of cells are labelled with different reagents, thereby allowing a comparison of the protein expression of the two sets of cells.

19. The method of claim 18, wherein the different reagents are distinguished on the basis of mass.

20. The method of claim 19, wherein the first set is labelled with a light isotopic label and the second set with a heavy isotopic label, or vice versa.

21. The method of claim 20, wherein the first set of cells is labelled with N-succinimidyl-2-(4-pyridylmethylthio)-acetate, and the second set of cells is labelled with N-succinimidyl-2-[4-(2,3,5,6-tetradeuterio-pyridyl)]-methylthioacetate.

22. The method of claim 19, wherein the two sets of cells are mixed before step (d).

23. The method of claim 19, wherein the protein-fragment mixture is analysed by
(i) scanning at 106 m/z, thereby generating a spectrum of the first set of cells,
(ii) scanning at 110 m/z, thereby generating a spectrum of the second set of cells, and
(iii) inverting the intensity values of the 110 m/z scan and adding them to the 106 m/z scan, thereby generating a difference spectrum.

24. The method of claim 19, wherein the protein-fragment mixture is analysed by
(i) neutral loss scanning at 105 m/z, thereby generating a spectrum of the first set of cells,
(ii) neutral loss scanning at 109 m/z, thereby generating a spectrum of the second set of cells, and
(iii) inverting the intensity values of the 109 m/z loss scan and adding them to the 105 m/z scan, thereby generating a difference spectrum.

25. The method of claim 23, wherein MS/MS-analysis is performed on peptides from a protein/polypeptide selected from the difference spectrum.

26. The method of claim 25, wherein the amino acid sequence is identified for at least one labelled peptide.

27. The method of claim 25, wherein an ion trap mass spectrometer is used.

28-29. (canceled)
